(19) United States
(12) Patent Application Publication
(10) Pub. No.: US 2011/0195054 A1
(43) Pub. Date: Aug. 11, 2011

(54) Preparation And Use Of Stromal Cells For Treatment Of Cardiac Diseases

(76) Inventors: Michael Cohen, West Orange, NJ (US); Ian K. McNiece, Coral Gables, FL (US)

(21) Appl. No.: 12/899,267

(22) Filed: Oct. 6, 2010

Related U.S. Application Data
(60) Provisional application No. 61/249,195, filed on Oct. 6, 2009.

(51) Int. Cl. A61K 35/12; C12N 5/071; A61P 9/00

(52) U.S. Cl. 424/93.7; 435/378; 435/325

(57) ABSTRACT

This invention is directed to the preparation and use of stromal cells for treatment of cardiac tissue.

This application claims the benefit of priority to U.S. Provisional Application No. 61/249,195, filed Oct. 6, 2009, which is herein incorporated by reference in its entirely.

### FIELD OF THE INVENTION

This invention relates to a method of preparing and a method of using stromal cells for the treatment of cardiac diseases.

### BACKGROUND OF THE INVENTION

Cardiovascular disease is the most common cause of death worldwide. The ability to augment weakened cardiac tissue would be a major advance in the treatment of heart disease and heart failure.

Stromal cells have the potential to differentiate to produce a variety of mesenchymal cell types (fibroblasts, bone, ligament, tendon, adipose tissue). Thus, stromal cells have gained interest as a potential treatment option for many diseases because they provide a renewable source of cells and tissues.

There remains a need for improved methods for preparing stromal cells, therapeutic compositions that include such stromal cells, and more effective treatment of cardiac diseases that use such stromal cells.

### SUMMARY OF THE INVENTION

This invention is drawn to a new and improved method for preparing stromal cells and the use of such prepared cells for the treatment of cardiac disease. In a preferred embodiment, the method of preparing stromal cells comprises:
a) enriching stromal progenitor cells from a bone marrow sample;
b) culturing said stromal progenitor cells in a culture vessel to expand stromal cells, wherein the stromal cells adhere to the culture vessel;
c) detaching the adhered stromal cells of step (b) and incubating them in an enhanced surface roller bottle; and
d) harvesting the stromal cells from said roller bottle.
   The stromal cells harvested in step (d) may be further cryopreserved until their use for patient therapy.

The stromal cells of the invention are preferably derived from bone marrow. More preferably, the stromal cells of the invention are expanded from multipotent mesenchymal stromal cells (stromal progenitor cells) obtained from bone marrow. The stromal cells may be referred to as mesenchymal stromal cells, bone marrow mesenchymal stromal stem cells, bone marrow stromal stem cells, bone marrow-derived mesenchymal stromal cells, multipotent mesenchymal stromal cells, or variations of these terms.

Another embodiment of the invention is drawn to the use of the stromal cells as prepared according to the invention for treatment of heart disease.

### BRIEF DESCRIPTION OF THE FIGURES

FIG. 1: A flow chart describing a preferred embodiment of a method of producing stromal cells according to the invention. The abbreviation “MNCs” refers to mononuclear cells, and the abbreviation “CFU-F” refers to fibroblast colony-forming, unit assay.

### DETAILED DESCRIPTION OF THE INVENTION

This invention is drawn to a new and improved method for preparing stromal cells and their use for the treatment of cardiac disease. In a preferred embodiment, the method of preparing stromal cells comprises:

a) isolating stromal progenitor cells from a bone marrow sample;

b) culturing said stromal progenitor cells in a culture vessel to expand the stromal cells, wherein the stromal cells adhere to the culture vessel;

c) detaching the adhered stromal cells of step (b) and incubating them in an enhanced surface roller bottle; and

d) harvesting the stromal cells from said roller bottle.

The bone marrow used as the source material for the inventive method may be autologous, allogeneic or xenogeneic. In a preferred embodiment, the bone marrow is allogeneic.

The bone marrow from which the stromal cells are isolated can be from a number of different sources, for example: plugs of femoral head cancellous bone pieces, samples obtained during hip or knee replacement surgery, or aspirated marrow obtained from normal donors and oncology patients who have marrow harvested for future transplantation. Preferred bone marrow sources are the iliac crest, femora, tibiae, spine, ribs or other medullary spaces in bone. Preferably, the marrow samples are screened for disease. Testing can include at least screening for anti-HIV-1/HIV-2, anti-HTLV I/II, anti-HCV, HBsAg (hepatitis B antigen), anti-HBc (hepatitis B core Ag) (IgG and IgM), and RPR (rapid plasma region) for syphilis.

The bone marrow sample is then prepared for cell culture and expansion. The culture and expansion process generally involves the use of specially prepared media that contains agents that allow for growth of stromal cells without differentiation and for the adherence of stromal cells to the plastic or glass surface of the culture vessel.

The harvested bone marrow may be washed, for example, in a PBS buffer (Baxter or Miltenyi) or Plasma-Lyte® A supplemented with 1% human serum albumin (HAS). The bone marrow is then processed to enriched stromal cell progenitor cells (multipotent mesenchymal stromal cells). For example, the marrow may be processed with Lymphocyte Separation Medium (LSM; specific gravity 1.077) (Lonza) to obtain the stromal progenitor cells. Various other separation means are known in the art that may be applied to this step.

The stromal progenitor cells may then be washed and sampled to determine the total number of viable nucleated cells.

The stromal progenitor cells are then cultured in order to expand stromal cells. Stromal cells are adherent and, accordingly, will adhere to the culture vessel and enable their separation from the remainder of the bone marrow cells. In a preferred embodiment, a stromal cell population as derived from the stromal progenitor cells (multipotent mesenchymal stromal cells) is expanded in complete media with antibiotics, with subsequent passages being in complete media without antibiotics.

The adherent stromal cells are then treated to remove them from the culture vessel. Adherent stromal cells can be detached from culture surfaces using a releasing agent such as trypsin, trypsin with EDTA (ethylene diaminetetra-acetic acid) (0.25% trypsin, 1 mM EDTA), or a chelating agent alone (e.g., EDTA or EGTA [ethylene glycol-bis-(2-amino ethyl ether) N,N-tetraacetic acid]). The selected releasing agent, after applying to and disrupting a confluent cell monolayer, can then be inactivated such as through the addition of complete medium or serum alone. The detached cultured stromal cells can be washed with complete medium for subsequent use.

The detached stromal cells are then incubated in an enhanced surface roller bottle. Examples of this type of roller bottle are available from Corning, Inc. (Corning, N.Y.). For example, the ribbed-surface Expanded Surface Polystyrene Roller Bottle product (850- and 1750-cm²) can be employed in practicing the invention. This roller bottle has a greater surface area for cell growth compared to standard roller bottles. Roller bottle rotation may be continuous or reciprocating. The stromal cells harvested from the roller bottle may be cryopreserved until their use for patient therapy.

After cell isolation and expansion according to the instant invention, the stromal cells can be genetically modified or engineered to comprise genes which express proteins of importance for striated muscle cell differentiation and/or maintenance. Transgenic sequences can be inserted into the genome of the stromal cells for stable gene expression, or expressed from a site ectopic to the genome (i.e., extra-chromosomal). Examples of genes for this purpose include growth factors (e.g., TGF-beta, IGF-I, FGF), myogenic factors (e.g., myoD, myogenin, Myf5, MRF), transcription factors (e.g., GATA-4), cytokines (e.g., cardiotrophin-1), members of the neuregulin family (neuregulin 1, 2 and 3) and homeobox genes (e.g., Csx, tinman, Nkx family). Also contemplated are genes that code for factors that stimulate angiogenesis and/or revascularization (e.g., vascular endothelial growth factor). Modes of introducing sequences to cells are well known in the art and include the provision of electroporation, cationic lipids and/or viral vectors (e.g., retrovirus, adenovirus, adeno-associated virus).

Stromal cells can be identified by specific cell surface markers that can be identified with unique monoclonal antibodies. The homogeneous stromal cell compositions of the instant invention can be obtained by positive selection of adherent bone marrow or periosteal cells which are free of markers otherwise specifically associated with hematopoietic cells and differentiated mesenchymal cells. These inventive stromal cell populations display epitopes specifically associated with stromal cells, have the ability to regenerate in culture without differentiating, and have the ability to differentiate into specific mesenchymal lineages when either induced in vitro or placed in vivo at the site of damaged tissue.

Listed below are certain reagents that are particularly useful for culturing and characterizing the stromal cells of the invention; these are available from several vendors such as Invitrogen (Carlsbad, Calif.):

Primary antibodies (preferably monoclonal): anti-CD73, anti-CD90, anti-CD105, anti-STRO-1, anti-CD11b (e.g., clone M1/70.15), anti-CD14 (e.g., clone RPA-M1), anti-CD19, anti-CD 34 (e.g., clone B1-3C5), anti-CD45 (e.g., clone HI30), anti-CD79α, anti-HLA-DR (e.g., clone LN-3), anti-Angiotensin 1 (AT1) and 2 (AT2) receptors. The multipotent mesenchymal stromal cells (stromal progenitor cells), which are used to produce the stromal cells of the invention, as initially enriched from bone marrow are plastic adherent, have fibroblast-like morphology (CFU-F), bear at least the stromal markers CD73 and CD105, and are negative for the haematopoietic markers CD14, CD34 and CD45.

Cell/Tissue Culture Media, Cell-Handling Reagents:

Dulbecco's Modified Eagle Medium (DMEM) (1×), liquid (low glucose). Contains 1,000 mg/L D-glucose and 110 mg/L sodium pyruvate. Without L-glutamine and phenol red.

Minimum Essential Medium Eagle, Alpha Modification (Alpha-MEM).

Dulbecco's Phosphate Buffered Saline (D-PBS) (1×), liquid. Without calcium, magnesium, or phenol red.

Dulbecco's Phosphate Buffered Saline (D-PBS) (1×), liquid. Contains calcium and magnesium, but no phenol red.

Phosphate Buffered Saline (PBS) (1×), liquid. Without calcium, magnesium, or phenol red.

Plasma-Lyte® A (pH 7.4). Can be used for cell washing procedures. Each 100 mL contains 526 mg NaCl, 502 mg of sodium gluconate, 368 mg of sodium acetate trihydrate, 37 mg of KCl, and 30 mg of MgCl₂.6H2O. Can be obtained from Baxter (Deerfield, Ill.).

CliniMACS® PBS/EDTA buffer saline, pH 7.2, (1 mM EDTA) (Miltenyi Biotec, Auburn, Calif.).

Fetal Bovine Serum (FBS). Can be gamma-irradiated. Use at final concentration of about 10-20%. Can obtain from manufacturers such as Invitrogen and Thermo Scientific (HyClone, Logan, Utah).

Human serum albumin (Baxter). Use at about 1% for processing cell. Use at about 2% for cryopreserving cells.

Gentamicin Reagent Solution (10 mg/mL), liquid.

Penicillin (10,000 units)/Streptomycin (10/mg/mL). Use at dilution of 1:100, for example.

GlutaMAX™-I Supplement

Hank's Balanced Salt Solution (HBSS) (1×), liquid. Contains calcium and magnesium.

Hank's Balanced Salt Solution (HBSS) (1×), liquid. Contains no calcium chloride, magnesium chloride, magnesium sulfate, or phenol red.

L-Glutamine-200 mM (100×), liquid. Use at about 2 mM, for example.

MEM Non-Essential Amino Acids Solution 10 mM (100×), liquid.

TrypLE™ Select (1×), liquid.

Trypsin-EDTA (0.25% Trypsin with EDTA.4Na) 1×. Can be gamma-irradiated.

Dimethyl sulfoxide (DMSO). Use at about 5% in cryopreserving cells.

HESpan®. Use at about 93% for cryopreserving cells. About 6% hetastarch in 0.9% NaCl. Can be obtained from B Braun Medical (Melsungen, Germany), for example.

Dimethyloxalylglycine (DMOG) may be supplemented to the above reagents to enhance stromal cell viability.

Growth Factors for Tissue Culture:

Basic Fibroblast Growth Factor (bFGF).

Bone Morphogenic Protein-2 (BMP-2).

Epidermal Growth Factor (EGF).

Insulin.

Laminin, Natural Mouse.

Fibronectin, Natural Human.

Insulin-Transferrin-Selenium-G Supplement (100×).

In a preferred embodiment, the stromal cells prepared according to the inventive method can be used in a therapeutic treatment. The stromal cells prepared according to the invention can be delivered to a patient, for example, for treating ischemia, hypoxia (e.g., placental hypoxia, preeclampsia), abnormal pregnancy, peripheral vascular disease (e.g., arteriosclerosis), transplant accelerated arteriosclerosis, deep vein thrombosis, cancers, renal failure, stroke, heart disease, sleep apnea, hypoxia during sleep, fetal hypoxia, smoking, anemia, hypovolemia, vascular or circulatory conditions which increase risk of metastasis or tumor progression, hemorrhage, hypertension, diabetes, vasculopathologies, surgery (e.g., per-surgical hypoxia, post-operative hypoxia), Raynaud's disease, endothelial dysfunction, regional perfusion deficits (e.g., limb, gut, or renal ischemia), myocardial infarction, stroke, thrombosis, frost bite, decubitus ulcers, asphyxiation, poisoning (e.g., carbon monoxide, heavy metal), altitude sickness, pulmonary hypertension, sudden infant death syndrome (SIDS), asthma, chronic obstructive pulmonary disease (COPD), congenital circulatory abnormalities (e.g., Tetralogy of Fallot) and erythroblastosis (blue baby syndrome). Thus, the invention can be directed to methods of treating diseases such as stroke, atherosclerosis, acute coronary syndromes including unstable angina, thrombosis and myocardial infarction, plaque rupture, both primary and secondary (in-stent) restenosis in coronary or peripheral arteries, transplantation-induced sclerosis, peripheral limb disease, intermittent claudication and diabetic complications (including ischemic heart disease, peripheral artery disease, congestive heart failure, retinopathy, neuropathy and nephropathy), or thrombosis. In a preferred embodiment, the stromal cells are administered for the treatment of a cardiac disease.

The stromal cells of the invention may be administered as a cell suspension in a pharmaceutically acceptable medium/carrier for injection, which can be local (i.e., directly into the damaged portion of the myocardium) or systemic (e.g., intravenous). Biological, bioelectrical and/or biomechanical triggers from the host environment may be sufficient, or under certain circumstances, may be augmented as part of the therapeutic regimen to establish a fully integrated and functional tissue.

The stromal cells of the invention can be administered in a biocompatible medium that comprises a semi-solid or solid matrix. A matrix selected for this purpose may be (1) an injectible liquid which polymerizes to a semi-solid gel at the site of the damaged myocardium, such as a collagen, a polylactic acid or a polyglycolic acid, or (ii) one or more layers of a flexible, solid matrix that is implanted in its final form, such as impregnated fibrous matrices. The matrix can be, for example, Gelfoam® (Upjohn, Kalamazoo, Mich.). A selected matrix serves to hold the stromal cells in place at the site of injury in the heart (i.e. functions as a scaffold of sorts). This, in turn, enhances the opportunity for the administered stromal cells to proliferate, differentiate and eventually become fully developed cardiomyocytes. As a result of their localization in the myocardial environment, either via a liquid medium of fibrous matrix, the administered stromal cells can integrate with the recipient's surrounding myocardium.

A non-limiting mechanism for such treatment involves differentiation of the stromal cells of the invention into cardiac muscle cells that integrate with the healthy tissue of the recipient to replace the function of dead or damaged cells, thereby regenerating the cardiac muscle as a whole. This is an important aspect of the invention given that cardiac muscle normally does not have the capability to repair itself.

A representative example of a stromal cell treatment and dose range is about 100 to 200 million cells. The frequency and duration of therapy would, however, vary depending on the degree of tissue involvement.

The following examples are included to demonstrate certain preferred embodiments of the invention for extra guidance purposes. As such, these examples should not be construed to limit the invention in any manner.

### EXAMPLES

### Example 1

The following method is a preferred embodiment for preparing stromal cells according to the invention.

1. Product Manufacturing—Components

1.1 Cells

1.1.1 Cell Source
The cell source for preparing this product was autologous or allogeneic bone marrow from normal donors.

1.1.2 Collection Methods
Bone marrow (about 30-60 mL) was aspirated from the posterior iliac crests of donors into heparinized syringes. Labeled syringes were transported at room temperature to a processing facility.

1.1.3 Donor Screening and Testing
Bone marrow donor screening followed standard transplant practices. Testing included, at a minimum, anti-HIV-1/HIV-2, anti-HTLV I/II, anti-HCV, HBsAg (hepatitis B antigen), anti-HBc (hepatitis B core Ag) (IgG and IgM), and RPR (rapid plasma region) for syphilis. Individuals testing positive for viruses were not be eligible for donating bone marrow.

2. Reagents

2.1 Tabulation of Reagents Used in Manufacturing
Table 1 contains a list of the reagents used in the manufacture stromal cells.

TABLE 1
Manufacturing reagents.

| | Concentration | | | |
|---|---|---|---|---|
| | used during | | | Reagent |
| Material | manufacturing | Vendor | Source | Quality |
| Lymphocyte Separation | 1x | Lonza | NA | Research |
| Medium | | | | grade |
| Alpha MEM | 1x | Gibco | NA | Research |
| | | (Invitrogen) | | grade |
| L-glutamine 200 mM | 2 mM | Gibco | NA | Research |
| | | (Invitrogen) | | grade |
| Penicillin/Streptomycin | 100 units/mL | Sigma or | NA | Research |
| 10,000 units Penicillin- | Penicillin | Gibco | | grade |
| 10 mg/mL Streptomycin | 100 μg/mL | (Invitrogen) | | |
| | Streptomycin | | | |
| Fetal Bovine Serum | 20% or 10% | Hyclone | Bovine | Research |
| (gamma-irradiated) | | | | grade |
| Trypsin with EDTA | 1x | SAFC or | Porcine | Research |
| (1X; 0.05% Trypsin) | | Gibco | | grade |
| (gamma-irradiated) | | (Invitrogen) | | |
| Plasma-Lyte ® A | 1x | Baxter | NA | Clinical |
| | | | | grade |
| CliniMACs ® | 1x | Miltenyi | NA | Clinical |
| PBS/EDTA buffer | | Biotec | | grade |
| PBS buffer | 1x | Baxter | NA | Clinical |
| (Ca⁺⁺ and Mg⁺⁺ Free) | | | | grade |
| Human serum albumin | 1% for | Baxter | Human | Clinical |
| | processing | | | grade |
| | 2% in Frozen | | | |
| | Product | | | |
| HESpan ® | 93% in Frozen | B Braun | NA | Clinical |
| | Product | Medical | | grade |
| DMSO | 5% in Frozen | Edwards | NA | Research |
| | Product | Lifescience | | grade |

Certificates of Analysis for reagents were obtained for all reagents.

2.3 Removal Method
Prior to infusion, the stromal cells were washed in a PBS buffer (either the Baxter or Miltenyi product) or Plasma-Lyte® A supplemented with 1% human serum albumin (HSA).

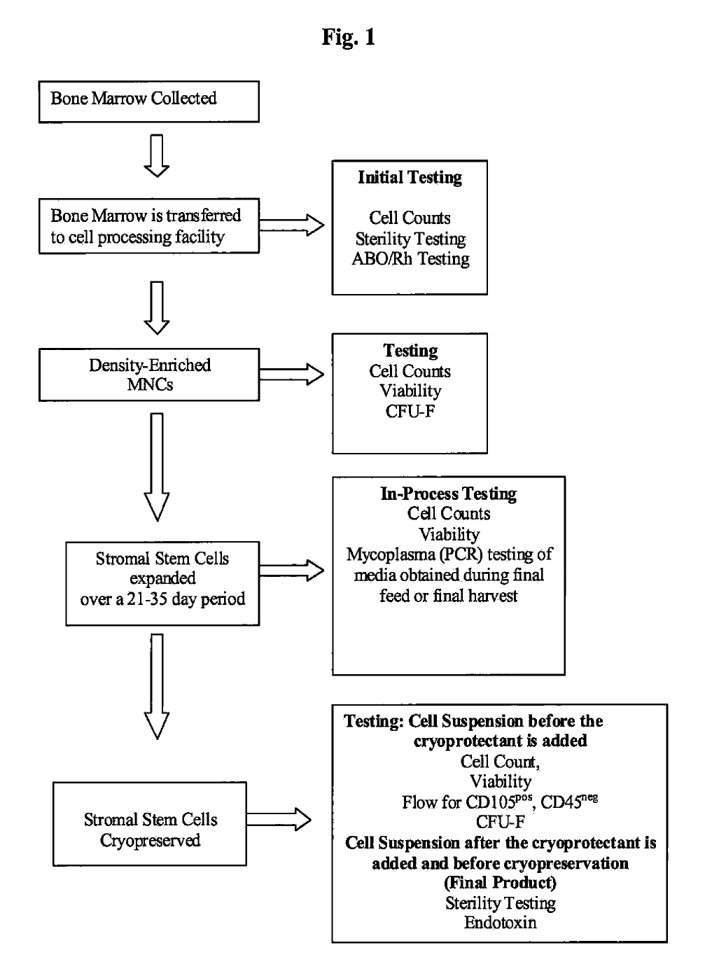

3. Product Manufacturing—Procedures
A schematic for the processing technique, which includes in-process and final testing, is shown in FIG. 1. All open manipulations in the production of the cell product were performed in a class 100 biological safety cabinet (BSC). All bags, syringes and reagents were sterile and disposable. All common laboratory equipment was cleaned between patient processing.

3.1 Stromal Progenitor Cell Enrichment
Bone marrow was processed using Lymphocyte Separation Medium (LSM; specific gravity 1.077) (Lonza) to enrich for stromal progenitor cells. The cells were diluted with Plasma-Lyte® A or PBS buffer and layered onto LSM using conical tubes. The enriched stromal progenitor cell preparation were washed with Plasma-Lyte® A or PBS buffer containing 1% human serum albumin (HSA). The washed cells were sampled to determine the total number of viable nucleated cells.

3.2 Stromal Cell Expansion
The enriched stromal progenitor cell preparation was initially cultured in a complete media with antibiotics consisting of alpha-MEM media supplemented with 2 mM L-glutamine, 20% fetal bovine serum (FBS), 100 units/mL penicillin, and 100 μg/mL streptomycin. Subsequent passages employed complete media without antibiotics. Stromal cell expansion was performed in flasks using a 37° C., 5% CO₂ humidified incubator. The stromal cells were detached from the culture vessels through trypsinization.

The P0 (passage 0) stromal progenitor cell preparation was cultured in ten T225 tissue culture flasks (surface area=225 cm²). When the cells in these flasks were confluent, the cells were passaged (i.e., passage 1) to six flasks resulting in sixty total P1 flasks. After incubation for approximately one week, the confluent P1 flasks were passaged to P2. Each flask was passaged to one 850-cm² enhanced surface roller bottle (Corning). After further incubation for approximately one week, when the stromal cells were confluent, each roller bottle was harvested. The harvested stromal cells were then cryopreserved as described below.

3.3 Stromal Cell Final Harvest and Cryopreservation
The stromal cells were counted and analyzed to determine total viable cells. Samples of these cells were taken as described in FIG. 1. The stromal cells were then suspended in a cryoprotectant consisting of HESpan® (6% hetastarch in 0.9% sodium chloride) supplemented with 2% HAS (human serum albumin) and 5% DMSO. The cells were subsequently aliquotted into cryopreservation bags. After cryopreservation using a control rate freezer, the frozen bags were placed into vapor phase nitrogen freezers where they were stored until issue.

3.4 Stromal Cell Preparation for Administration (Final Formulation)
Frozen stromal cells were thawed in a 37±1° C. water bath. In a BSC, the thawed cell suspension was transferred to conical tubes and slowly diluted with a PBS buffer or Plasma-Lyte® A supplemented with 1% human serum albumin. The diluted suspension was centrifuged and the resulting cell pellet, after removal of supernatant fluid, was suspended in buffer solution. The cells were counted to determine viability. The cells were centrifuged and the resulting cell pellet was resuspended in dilution buffer (PBS or Plasma-Lyte A® with 1% HSA) to the required cell concentration. This cell preparation was ready for administration to a patient.

4. Validation of Allogeneic Stromal Cell Manufacture
Tables 2 and 3 list details of a typical manufacturing run as described above.

TABLE 2
Validation Product Release Test Results.

| Assay | Sample Analyzed | |
|---|---|---|
| Sterility: | Final Product | Negative |
| aerobic/anaerobic/fungal | | |
| Mycoplasma | Cells in Conditioned | Negative |
| (PCR detection) | Media prior to | |
| | harvest | |

TABLE 3
Characterization of Stromal Cell Expansion.

| | |
|---|---|
| Starting total cell no. (×10⁶) | 656 |
| Post-Ficoll ® total cell no. (×10⁶) | 150 |
| Number of P0 flasks | 10 |
| Cells per P0 flask (×10⁶) | 15 |
| P0 total harvest (×10⁶) | 126 |
| Number of P1 flasks | 60 |
| No of days in culture (P1) | 14 |
| P1 total harvest (×10⁶) | 1096 |
| Number of P2 roller bottles | 60 |
| P2 total harvest (×10⁶) | 5,480 |
| P2 Sterility result | Negative |
| P2 Viability | >96% |
| P2 Final flow CD105⁺/CD45^{neg} | >95% |
| P2 Mycoplasma Culture | Negative |

5 Product Testing

5.1 Sample Procurement
Shown in Table 4 are the tests performed at each step in the process.

TABLE 4
Assay Test Sample List.

| Sample | Assay/Method |
|---|---|
| Starting cells | Automated cell count |
| | Sterility |
| | ABO/Rh testing |
| Stromal progenitor cell- | Automated cell count |
| enriched preparation | Viability |
| | CFU-F |
| Stromal cells at each passage | Manual cell count |
| | Viability |
| Stromal stem cells and | Mycoplasma (PCR) |
| conditioned media | |
| (final feed or final harvest) | |
| Stromal cells prior to the | Manual cell count |
| addition of cryoprotectant | Viability |
| | Flow cytometry: CD105^{pos} and CD45^{neg} |
| | CFU-F |
| Stromal cells after the | Sterility |
| addition of cryoprotectant | Endotoxin |
| (final product) | |
| Stromal cells after thawing, | Manual cell count |
| prior to injection or infusion | Viability |
| | Endotoxin |
| | Sterility |
| | Gram stain |

The use of enhanced surface roller bottles as described above provided enhanced expansion (increased cell number) of stromal cells that did not exhibit significant morphological changes compared to stromal cells prepared by standard methodologies. Further, the utilization of the roller bottles allowed for a faster replication process when associated with variant environmental ambient temperature. Interestingly, the stromal cells obtained by this process also exhibited lower expression of interleukins-1 and -6 (IL1 and IL6). It was also observed that when the roller bottles were subjected to alternating directional rotation (i.e., reciprocating movement) during stromal cell incubation, there were more non-adherent cells compared to static flask cultures.

### Example 2

Intracoronary Infusion of Stromal Cells to Patients with Chronic Coronary Ischemia.

Allogeneic or autologous human stromal cells may be prepared from one or more bone marrow aspirates according to the method described in Example 1. Transplantation of stromal cells into a patient would be performed as follows. After thawing, the stromal cells (100-200 million cells) would be administered by a surgeon to the patient by catheter-based injection into and/or about the periphery of the ischemic lesion(s) of the heart. Postoperative follow-up patient care would include evaluating the effects of stromal cell engraftment on lesion size and cardiac function.

### Example 3

The stromal cells from 60 cc of bone marrow are isolated by density centrifugation and seeded into 60 T185 cm² flasks. The flasks are incubated at 37° C. in 5% CO₂.

The media is changed twice weekly in the flasks until the flasks are confluent (approximately 3 weeks). When they reach confluency, the flasks are harvested by trypsin treatment and frozen in liquid nitrogen in 10 bags containing 15 to 25 million stromal stem cells per bag (P0).

Each bag is thawed as needed and seeded into 10×T185 cm2 flasks (P1) containing 1 to 3 million stromal stem cells per flask. After a week confluent flasks are harvested using trypsin treatment and the cells seeded into 60×T185 cm2 flasks (P2). When these flasks are confluent they are harvested and the stromal stem cells seeded into a final number of 1.80×T185 cm2 flasks (P3). When these flasks are confluent, the cells are harvested and frozen in bags in LN2 at 50 million cells per bag. Typically this will result in approximately 1.5 billion stromal stem cells for each P3 bag of stromal stem cells.

This summarizes the process which generates 15 billion stromal stem cells at P3 in approximately 6 to 7 weeks. The expended stromal stem cells maintain their morphological structure that is identical to the original cells.

What is claimed is:

1. A method for preparing stromal cells comprising:
a) enriching stromal progenitor cells from a bone marrow sample;
b) culturing said stromal progenitor cells in a culture vessel to expand stromal cells, wherein the stromal cells adhere to the culture vessel;
c) detaching the adhered stromal cells of step (b) and incubating them in an enhanced surface roller bottle; and
d) harvesting the stromal cells from said roller bottle.

2. A method of treating cardiac tissue comprising administering to a patient stromal cells prepared according to claim 1.

3. The method of claim 2, wherein the stromal cells are administered directly to the heart of an individual with cardiac trauma.

4. A therapeutic composition comprising stromal cells prepared according to the method of claim 1, wherein said stromal cells are present in an amount effective to promote the regeneration of cardiac tissue.
